(19) United States
(12) Patent Application Publication
(10) Pub. No.: US 2006/0212122 A1
(43) Pub. Date: Sep. 21, 2006

(54) Intervertebral disk prosthesis

(76) Inventor: Fiorella Perera, Pfaffikon (CH)

Correspondence Address: BUCHANAN, INGERSOLL & ROONEY PC, POST OFFICE BOX 1404, ALEXANDRIA, VA, 22313-1404, US

(21) Appl. No.: 10/564,279

(22) PCT Filed: Jul. 11, 2004
(86) PCT No.: PCT/CH04/00442

§ 371 Date: Jan. 11, 2006

(30) Foreign Application Priority Data
Jul. 12, 2003 (CH) 1213/03
Jun. 30, 2004 (CH) 1100/04

(51) Int. Cl. A61F 2/44

(52) U.S. Cl. 623/17.14

(57) ABSTRACT

The invention relates to an intervertebral disk prosthesis formed by an upper part and a lower part. The top face of the upper part and the bottom face of the lower part are provided with essentially convexly curved areas. The bottom face of the upper part is embodied as a convexly or concavely shaped spherical area while the top face of the lower part is provided with a concavely or convexly shaped spherical area. The upper part and the lower part rest against each other in an at least partly jointless manner, movability of the two vertebrae being ensured by moving the spherical areas relative to each other. Wear effects are kept low by providing at least one spherical area with a coating. Loss of blood is expected to be reduced while operating times and recovery times are expected to be shortened and the risk is expected to decrease if the inventive prosthesis is inserted retroperitoneally. Also disclosed is a method for producing a correctly fitting intervertebral disk prosthesis, resulting in perfect adaptation to the anatomy of the vertebral bodies.

The invention relates to an intervertebral disk prosthesis as claimed in claim 1 and a process in this respect as claimed in claim 23.

The intervertebral disk behaves like a “natural ball bearing” and enables the vertebrae to move in different directions since the joint has elastic properties. The intervertebral disk is used as a buffer for the forces which move up and down on the human spinal column. In normal intervertebral disk function the joint faces on either side of the spinous processes are kept apart from one another at the correct distance. The intervertebral disk provides for the foremen to be large enough so that the nerve is not hindered.

Two ligaments run on the front and back of the actual vertebral body. On the back the intervertebral disk and the fibrous ligament merges with the edges of the vertebrae located above and below, thus an anchor or a type of brace for the intervertebral disk and the two interconnected vertebra forms. On the front the intervertebral disk merges with the ligament, but not with the anterior vertebral edge. The ligament is pulled up and down and is very securely connected to the front of the vertebral bodies, but recesses the vertebral bodies. This variation in the anatomical attachment of the intervertebral disk to the vertebrae determines the function of the intervertebral disk. The type of attachment creates a potential space between the intervertebral disk and the vertebra on the front, but not on the back. If specifically two types of tissue in the body are not securely connected to one another, a potential anatomic intermediate space forms between them. In a movement which compresses the vertebrae with force, a large part of the force is directly to the rear. Since the intervertebral disk is designed to relay the force, it would undoubtedly move with the force if it were freely movable. The intervertebral disk is however connected to the anterior longitudinal ligament which behaves like the chord of an arc. As the cord draws the sagitta, the longitudinal ligament draws the intervertebral disk back.

It is known that the intervertebral disks can be crowded or that the inner nucleus pulposus can emerge through cracks in the connective tissue-like, cartilaginous outer annulus fibrosus. In this case the intervertebral disk can partially squeeze into the intervertebral foramina or the vertebral canal. Moreover this prolapse can be dorsal medial or lateral. These prolapses occur most often on the L4-L5-S1 and C6-C7 vertebrae. If these prolapses are not treated, irreversible compressive damage of the nerve roots (foramina) or cross sectional lesions occur. If symptomatic physiotherapy, for example remedial gymnastics or massage, should not be successful, the intervertebral disk must be surgically removed.

WO 01/01893-A1 (Spine Solution Inc.) discloses a 3-part intervertebral implant which consists of an upper part, a lower part and a joint insert which can be inserted between them. The joint insert has a spherical support surface which allows a certain swivelling capacity of the upper part and lower part and thus also allows a swivelling capacity of the adjacent vertebral bodies. The comb-like projections which are mounted on the upper part and lower part are used for anchoring in the corresponding vertebral bodies in which the receivers for them must be incorporated; this is not only complex, but represents a weakening of the vertebral bodies. The necessary unravelling of the ligament results in stability losses of the spinal column. Moreover it is disadvantageous that the intervertebral implant consists of 3 parts.

Furthermore, U.S. Pat. No. 4,349,921 (Kuntz) discloses a one-part or two part intervertebral disk prosthesis which is provided with grooves transversely to the insertion direction and on one side has a flange or projections. This flange prevents overly deep penetration of the prosthesis and injury to spinal nerves by its resting on the vertebral edges. Furthermore, at least partial mobility of the vertebrae will be ensured. The disadvantage is the unwanted migration of the prosthesis in the intervertebral disk space since it does not provide for additional attachment to the vertebral bodies.

The object of the invention is to devise an intervertebral disk prosthesis which is used as an intervertebral disk replacement and which continues to ensure the mobility of the two adjoining vertebrae. Another object is a process for doing this.

As claimed in the invention, this object is achieved with an intervertebral disk prosthesis according to the wording as claimed in claim 1 and a process for this according to the wording as claimed in claim 23.

The invention is described below using figures.

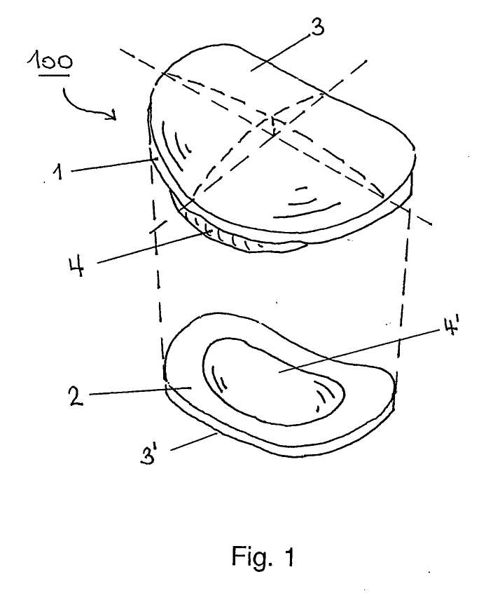

FIG. 1 shows a perspective exploded view of an intervertebral disk prosthesis

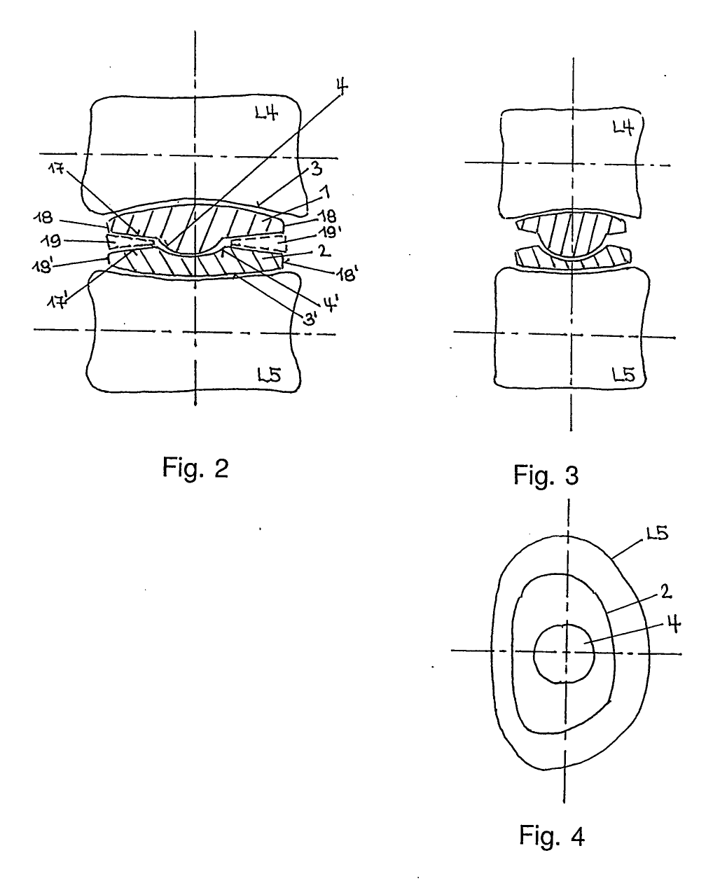

FIG. 2 shows a section of the intervertebral disk prosthesis as shown in FIG. 1

FIG. 3 shows a side view to FIG. 2

FIG. 4 shows a top view to FIG. 2

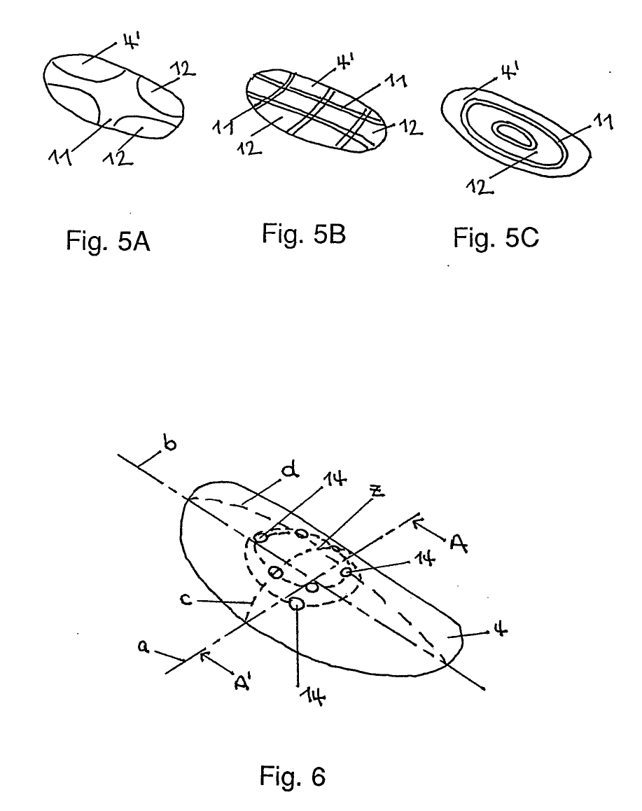

FIG. 5A-5C shows a spherical surface with different types of coatings

FIG. 6 shows a spherical, convexly arched surface with circular openings

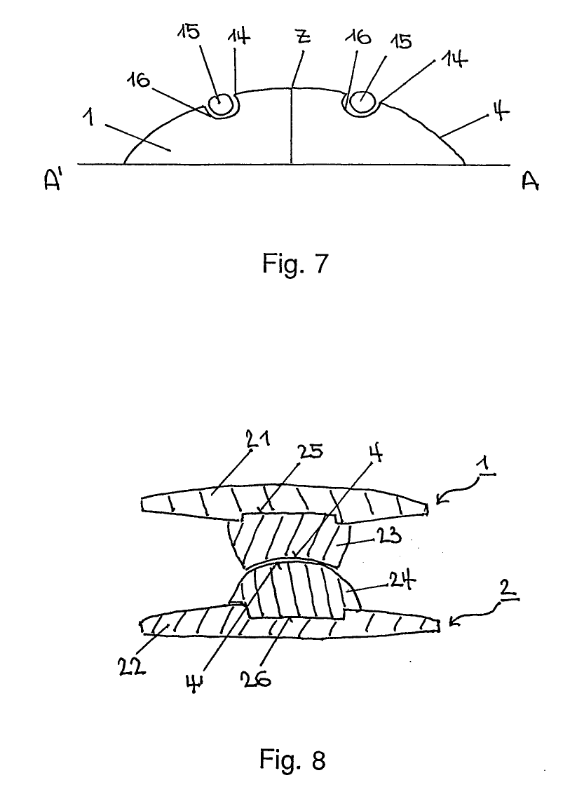

FIG. 7 shows a section A-A to FIG. 6

FIG. 8 shows a subdivided intervertebral disk prosthesis in a section.

The intervertebral disk prosthesis as claimed in the invention is used between two vertebral bodies of the spinal column, is implanted there and is used as an intervertebral disk replacement. Consequently, by it the original intervertebral disk height is reached again, the nerve roots of the foramina return to their original size, and mobility is restored. With this prosthesis none of the vertebral bodies which lie on top of one another are stiffened any more; this is especially advantageous compared to known surgical techniques.

This prosthesis is implanted retroperitoneally. Thus spinal nerves, spinous processes and articular processes are no longer damaged or removed. All ligaments/bands (flavum, capsulary, interspinous, supraspinous, intertransverse and the two longitudinal ligaments (anterior and posterior longitudinal ligaments)) are preserved. Muscles are not damaged any more. This means that the tension and the function of these muscles and ligaments enable posture and flexible activity which maintains healthy stability and curvature of the spinal column.

This new and simple retroperitoneal insertion greatly shortens the surgery time, blood loss is less, and there is no danger of damage to the dural sack and the spinal nerves.

FIG. 1 shows in an exploded view an intervertebral disk prosthesis 100 which consists of an upper part 1 and a lower part 2. The upper part 1 on its top has an essentially convexly curved surface 3, while its bottom has at least in part an essentially spherical surface 4. The lower part 2 on its bottom has an essentially convexly curved surface 3′, while its top has at least in part a spherical or cap-like surface 4′ which is essentially pointed down. As shown, the surface 4 is convexly shaped, while the surface 4′ is concavely shaped. The surfaces 4, 4′ can however also be shaped oppositely, specifically the surface 4′ can be concave and the surface 4′ can be convex. The spherical surfaces 4, 4′ have an essentially identical spherical radius so that the upper part 1 and the lower part 2 can at least essentially seamlessly adjoin one another and thus form a two-part intervertebral disk prosthesis.

The parts 1, 2 move on the spherical surfaces 4, 4′ in which the mobility of the intervertebral disk prosthesis is based.

The convexly curved surfaces 3, 3′ are chosen in their shape such that they are adapted to the anatomical requirements of the intervertebral space. They are generally slightly convexly curved, but in a boundary case can also be made planar.

The spherical surfaces 4, 4′ generally cover a wide area of the bottom and top of the parts 1,2. In the boundary case they cover the entire bottom and top. Spherical can be strictly geometrical or with minor deviations, especially for the convex part; this can be quite advantageous.

The spherical radii of the spherical surfaces 4, 4′ are either exactly the same or allow minor deviations, especially for the radius of the convex surface; this in turn can be advantageous. It follows from this that part 1 and part 2 either adjoin one another strictly seamlessly, or which is the case for slightly different spherical radii, have a more or less pronounced seam in the outer regions of the surfaces 4, 4′. The expression “essentially seamlessly” should be understood in this respect.

Possible materials for the parts 1, 2 are plastics, carbon-fiber reinforced plastics, metals and metal alloys, and ceramic materials:
plastics such as polyether ether ketones (PEEK), polyether ketone ether ketone ketones (PEKEKK) and polysulfones (PS) are preferably used, and especially preferably as a composite material, carbon fiber-reinforced composites of polyether ether ketone (CFK/PEEK) and polyether ketone ether ketone ketones (CFK/PEKEKK) which are also known under the names ULTRAPEK and OSTAPEK.
Metals or metal alloys are stainless, or rust-resistant metals and their alloys (DIN ISO Standard 5832-1), preferably titanium and its alloys, such as for example titanium alloy Ti6-A14-V according to DIN ISO Standard 5832-3,or Co-Cr-Ni alloys according to DIN ISO Standard 5832-4.

Ceramic materials include zirconium ceramics, Al₂0₃ bioceramic and hardened ceramic (silicon nitride).

The parts 1, 2 can also consist of different materials. The formation of a sliding pairing of materials with reference to adjoining surfaces 4, 4′ in order to satisfy the requirements for compatibility, wear and service life is revolutionary. If the same materials are used, generally the surface of one part is provided with an additional coating, as is described later.

The parts 1, 2 can also be made as composite parts. Thus a first part of the composite with surface 3, 3′ can consist for example of a Co-Cr-Ni alloy in conjunction with a second part of the composite with a surface 4, 4′ of a ceramic.

FIG. 2 shows one view of the intervertebral disk prosthesis as shown in FIG. 1 between two vertebral bodies.

Part 1 and part 2 with the spherical surfaces 4, 4′ for which a spherical radius R is shown adjoin one another. The convexly curved surfaces 3, 3′ adjoin the vertebral bodies L4, L5, the surfaces adjoining the vertebral bodies being apparent. The convexly curved surfaces 3, 3′ are made large that they ensure loading as uniform as possible over the entire surface.

The surfaces 4, 4′ only partially stress the bottom and top of the parts (1) and (2). This yields zones 17, 17′ on the bottom and the top of the parts (1) and (2). These zones are bordered on the one hand by the surfaces 4, 4′ and on the other by the edges 18, 18′ of the intervertebral disk prosthesis. The zones 17, 17′ define a free space 19 and 19′, as is shown in the undeflected state. This free space becomes smaller on one side by the deflection of the two prosthesis parts. It can essentially disappear at maximum deflection, then the two edges 18, 18′ adjoin one another on one side. The geometry of the zones 17, 17′ is critical, since ultimately they define or limit the mobility of the vertebral bodies against one another.

Materials for coatings of the convexly curved surfaces 3, 3′ can be a hydroxyl-apatite ceramic (HAK) coating, a hydroxyl-apatite ceramic (HAK) coating with beaten-on tantalum or titanium, or a tri-calcium phosphate (TCP) coating, by which the long-term properties of the intervertebral disk prosthesis are benefitted.

The spherical surfaces 4, 4′ of the intervertebral disk prosthesis are advantageously entirely or at least partially provided on one side at a time with another coating which efficiently supports the sliding or friction properties of part 1 in or on part 2. That is, good sliding properties are achieved in this way and thus wear is kept low to the benefit of longer service life.

Materials for this coating can be plastics such as polyethylene and polypropylene, preferably a high pressure-process polyethylene (HD-PE). Furthermore, coatings of ceramic material are used.

FIG. 3 shows a side view to FIG. 2. The parts 1, 2′ and the two vertebral bodies L4, L5 are recognizable.

FIG. 4 shows a top view to FIG. 2 without the vertebral body L4 and without part 1. The vertebral body L5 and part 2 with the oval, spherical surface 4 are recognizable.

FIGS. 5A-C show the spherical surface with different types of coatings in a perspective.

The coatings 11 of the spherical surfaces 4, 4′ then cover them entirely or at least partially. FIGS. 5A-5C show different possibilities for partial covering of the spherical surfaces.

In FIG. 5A the coating is made cruciform and accordingly does not cover areas 12 on the edges of the spherical surfaces 4 or 4′. The surface pressure on this cruciform coating is accordingly greater than for a coating which covers the entire surface.

In FIG. 5B the coating is made strip-shaped and accordingly does not cover the areas 12 between the strips. The strips are preferably overlapping, i.e they cross one another and form a network-like structure. Parallel running strips are also possible.

In FIG. 5C the coating is made in concentric strips and accordingly does not cover the areas 12 between the strips. For each concentric strip here the coating can be chosen to have different thicknesses, by which the different surface pressure from the outside to the inside or from the inside to the outside is taken into account.

FIG. 6 shows a perspective of a spherical, concavely arched surface with circular openings. The surface 4 rises from the plane which is formed by lines a, b, and constitutes a convexly arched surface; this is recognizable by the broken subsidiary lines c, d. The subsidiary lines c, d cross one another at a point Z which forms the center for at least one concentric circle 13. Along the circumference of this circle circular openings 14 are made which are designed to guide balls (not shown); this is explained later. For the sake of clarity, only two concentric circles 13 and in the second circle only one circular opening 14 are shown. Several circles are conceivable with circular openings distributed on their circumference. Advantageously the openings are uniformly distributed. Of course there can also be a circular opening 14 in the center Z.

FIG. 7 shows a corresponding section A-A′ to FIG. 6. On the concavely arched surface 4 of the part 1 circular openings 14 which are located on the circumference of the circle with a center Z can be recognized. Added balls 15 which are located in spherical cavities 16 and which are pivotally supported in them project out of the circular openings 14. Thus the balls acquire the fiction of a ball bearing, since the adjoining surface of the second prosthesis part which is not shown is supported by the balls 15 and moves on them relative to the surface 4. This yields the function of a multidimensional, ball-supported arrangement. This results in mobility which is defined not only in one plane, but which can take place in any planes.

Of course the spherical cavities 16 can alternatively also be provided in a concavely arched surface 4′. In turn, the adjoining, now convex surface of the second prosthesis part moves supported on the balls 15 relative to the concavely arched surface.

Balls of the ceramic material silicon nitride are preferably used. These balls have an especially hardened surface.

FIG. 8 shows an intervertebral disk prosthesis with subdivided parts 1, 2 in a section. The parts 1, 2 are divided and on the vertebra side have parts 21 and 22 in which the adjoining parts 23 and 24 are embedded in recesses 25 and 26 of parts 1, 2. Division proves advantageous in a freer choice of the materials with respect to compatibility on the vertebra side and the sliding pairing of the adjoining parts 23, 24. More extensive division of the parts 1, 2′ into more than two parts is likewise conceivable. During deflection the uniform support on the adjoining surfaces 4, 4′ for any degree of deflection is advantageous.

Of course the structure of the intervertebral disk prosthesis can be altered within wide limits within the framework of this invention. Thus, for example replacement of part 1 with part 2 is quite possible; this is equivalent to using the intervertebral disk prosthesis “upside down”.

Structures of the described type are self-centering between the vertebral bodies. Therefore any fastening elements on the two parts 1 and 2 can be abandoned. With a screw connection not only is the attachment of the prosthesis parts to the vertebral bodies known to be achieved, but also unwanted states of tension are produced by the screw connection and they only partially diminish with time and therefore are a problem. Moreover the holes for holding the screws reduce the stability of the healthy spongiosa bone.

The advantages of the intervertebral disk prosthesis as claimed in the invention thus arise due to the fact that after completed surgery the mobility of the vertebral bodies is essentially preserved, that during the surgery lower blood losses occur, that less surgical time is necessary and that healing times are shorter with lower risk.

The examples described below provide some insight into the diversity of the configuration of an intervertebral disk prosthesis and its enumeration should not be considered exhaustive in any case.

### EXAMPLE 1

An intervertebral disk prosthesis as shown in FIG. 1 has a spherical radius of the surfaces 4, 4′ of 33 mm. The parts 1, 2′ are produced from carbon fiber-reinforced composite polyether ketone ether ketone ketone (CFK/PEKEKK). The convexly curved surfaces 3, 3′ have a tricalcium phosphate (TCP) coating. The spherical surface 4′ is provided in its entirety with a 0.6 mm thick coating of high pressure-process polyethylene (HD-PE).

### EXAMPLE 2

An intervertebral disk prosthesis essentially as shown in FIG. 1 has a spherical radius of the surfaces 4, 4′ of 30 mm. The parts 1, 2 are produced from carbon fiber-reinforced composite polyether ether ketone (CFK/PEEK). The convexly curved surfaces 3, 3′ have a hydroxyl-apatite ceramic (HAK) coating. The spherical surface 4 has a 0.45 mm thick coating of polyethylene (PE) with concentric strips as shown in FIG. 5C Thus the surface 4 is only partially covered (60%).

### EXAMPLE 3

An intervertebral disk prosthesis essentially as shown in FIG. 1 has a spherical radius of the surfaces 4, 4′ of 32 mm. The part 1 is made as a composite part. The surface 3 is made from a Co-Cr-Ni alloy to which as the composite an A1₂0₃ bioceramic which forms essentially the surface 4 is attached. The part 2 consists of a Co-Cr-Ni alloy with a spherical surface 4′ which has a 0.5 mm thick coating of high pressure-process polyethylene (HD-PE) which is applied in the shape of a cross as shown in FIG. 5A. Thus the surface 4′ is only partially covered (80%). The convexly curved surfaces 3, 3′ have a hydroxyl-apatite ceramic (HAK) coating with beaten-on tantalum.

### EXAMPLE 4

An intervertebral disk prosthesis essentially as shown in FIG. 1 has a spherical radius of the surfaces 4, 4′ of 28.5 mm. The part 1 is made as a composite part. The surface 3 consists of a titanium alloy to which a hardened ceramic as the composite which forms essentially the surface 4 is attached. In the hardened ceramic of the part 1 cavities 16 are formed in which there are balls of silicon nitride which project out of the circular openings 14. The convexly curved surfaces 3, 3′ have a hydroxyl-apatite ceramic (HAK) coating. The part 2 consists of a titanium alloy with a spherical surface 4′ which has a 0.5 mm thick coating of high pressure-process polyethylene (HD-PE) over the entire surface. This intervertebral disk prosthesis can accordingly be called “multidimensional ball bearings”.

### EXAMPLE 5

An intervertebral disk prosthesis essentially as shown in FIG. 8 has a spherical radius of the surfaces 4, 4′ of 39 mm. The part 1 is subdivided into parts 21 and 23 and the part 2 into parts 22 and 24. The parts 21, 2′2 are made of titanium and on the vertebra side have a hydroxyl-apatite ceramic (HAK) coating with beaten-on tantalum. The parts 23, 2′4 consist of a zirconium ceramic.

Furthermore, a process which belongs to such an intervertebral disk prosthesis is described. Prior to the surgery, the spinal column in the area around the damaged intervertebral disk, especially the vertebral bodies, is measured by means of a scanning process, a 3-D scanning process, or a similar, equivalent process. In doing so characteristic data of those surfaces of the vertebral bodies which the intervertebral disk prosthesis adjoins or rests upon are determined. Using the height of the adjacent intact intervertebral disks or the distance between the adjacent intact vertebral bodies, the original height of the damaged intervertebral disk (intervertebral height) is deduced or this height is determined by extrapolation. This height corresponds to the height of the intervertebral disk prosthesis which is composed of parts 1, 2′, 23 and 24. All the characteristic data are obtained from the raw data of the scanning process by data reduction which will not be detailed. It is important that the set of characteristic data is used for producing the intervertebral disk prosthesis, and for this purpose is generally sent electronically to a production center and used to produce an intervertebral disk prosthesis tailored to the patient. An intervertebral prosthesis which has been fabricated in this way is perfectly matched to the vertebral bodies. It is self-centering, does not require additional fixing, and under the best assumptions can “grow on or in”. Migration is thus precluded. Moreover the adjoining vertebral bodies are not weakened or damaged by a screw mounting; this could lead to destabilization. It is important that this process renders the site of the surgery independent of place and time. The determination of the characteristic data in the scanning process can take place beforehand, i.e. at almost any time before the surgery, while production of the intervertebral disk prosthesis, or parts of it, takes place at a location which is completely independent of the location of determination and surgery.

1. Intervertebral disk prosthesis comprising an upper part and a lower part, wherein the intervertebral disk prosthesis is formed from an upper part and a lower part, the top of the upper part and the bottom of the lower part having essentially convexly curved surfaces, wherein the lower side of the upper part has at least partially an essentially convexly or concavely shaped spherical surface while the upper side of the lower part has an essentially concavely or convexly shaped spherical surface, the spherical surfaces having an essentially identical spherical radius so that the upper part and the lower part adjoin one another at least partially essentially seamlessly and thus form a two-part intervertebral disk prosthesis, and wherein the mobility of the two vertebrae is dictated by the motion of the spherical surfaces against one another.

2. Intervertebral disk prosthesis as claimed in claim 1, wherein the convexly curved surfaces have a first coating, the surfaces being entirely or at least partially covered.

3. Intervertebral disk prosthesis as claimed in claim 2, wherein the first coating is a hydroxyl-apatite ceramic (HAK) coating, a hydroxyl-apatite ceramic (HAK) coating with beaten-on tantalum or titanium, or a tricalcium phosphate (TCP) coating.

4. Intervertebral disk prosthesis as claimed in claim 1, wherein the spherical surfaces have entirely or at least partially another coating on one side at a time.

5. Intervertebral disk prosthesis as claimed in one of claim 1, wherein the spherical surfaces consist of different material.

6. Intervertebral disk prosthesis as claimed in claim 1, wherein one of the parts with a convexly curved or arched surface has cavities in which balls are pivotally placed which project on the circular openings of the surfaces and are designed for sliding on the adjoining concavely curved surface.

7. Intervertebral disk prosthesis as claimed in one of claim 1, wherein one of the parts with a concavely curved or shell-like surface has cavities in which balls are pivotally placed which project on the circular openings of the surfaces and are designed for sliding on the adjoining convexly curved surface.

8. Intervertebral disk prosthesis as claimed in claim 6 wherein the balls consist of a ceramic material, preferably of zirconium ceramic, Al₂0₃ bioceramic or hardened ceramic (silicon nitride).

9. Intervertebral disk prosthesis as claimed in claim 4, wherein the other coating consists of polyethylene and polypropylene, preferably of high pressure-process polyethylene (HD-PE).

10. Intervertebral disk prosthesis as claimed in claim 4, wherein the other coating consists of a ceramic material, preferably of a bioceramic.

11. Intervertebral disk prosthesis as claimed in claim 9 wherein the other coating is cruciform, network-like, or in concentric rings.

12. Intervertebral disk prosthesis as claimed in claim 1, wherein the upper and lower parts consist of plastic, preferably of polyether ether ketone (PEEK), polyether ketone ether ketone ketone (PEKEKK) or of polysulfone (PS) or a composite material, preferably carbon fiber-reinforced composite of (CFK/PEEK) and (CFK/PEKEKK).

13. Intervertebral disk prosthesis as claimed in one of claim 1, wherein the parts consist of titanium, a Ti alloy or of a Co-Cr-Ni alloy.

14. Intervertebral disk prosthesis as claimed in one of claim 1, wherein the upper and lower parts consist of a ceramic material, preferably of zirconium ceramic, A1₂O₃ bioceramic or a hardened ceramic (silicon nitride).

15. Intervertebral disk prosthesis as claimed in claim 1, wherein of the parts at least one consists of a composite material.

16. Intervertebral disk prosthesis as claimed in claim 1, wherein the upper part and the lower part consist of different material.

17. Intervertebral disk prosthesis as claimed in claim 1, wherein the upper part and the spherical surface as well as part and the spherical surface consist of different material.

18. Intervertebral disk prosthesis as claimed in claim 1, wherein the upper and lower parts are interchangeable.

19. Intervertebral disk prosthesis as claimed in claim 1, wherein it is self-centering between the vertebral bodies.

20. Intervertebral disk prosthesis as claimed in claim 1, wherein the upper part and the lower part adjoin one another at least partially seamlessly.

21. Intervertebral disk prosthesis as claimed in claim 1, wherein it has free spaces which are bordered by zones on the bottom and top of the upper part and the lower part the free spaces essentially disappearing on one side at a time at maximum deflection of the parts.

22. Intervertebral disk prosthesis as claimed in claim 1, wherein the part and/or the lower part is divided into at least two parts.

23. Process for producing an intervertebral disk prosthesis as claimed in claim 1, wherein the spinal column is measured beforehand in the area around the damaged intervertebral disk and especially the vertebral bodies by means of a scanning process, characteristic data being determined and wherein the intervertebral disk prosthesis is designed based on the characteristic data and in this way perfect matching to the anatomy of the vertebral bodies is achieved.

24. Process as claimed in claim 23, wherein the support surfaces of the vertebral bodies are measured and the convexly curved surfaces are designed by means of the characteristic data.

25. Process as claimed in claim 23, wherein the heights of the adjacent intact intervertebral disks are measured and wherein the height of the intervertebral disk prosthesis is engineered by means of the characteristic data which have been determined by extrapolation.

26. Process as claimed in claim 22, wherein measurement, construction and surgery are carried out independently of one another in terms of time and space.
